(19) United States
(12) Patent Application Publication
(10) Pub. No.: US 2018/0177777 A1
(43) Pub. Date: Jun. 28, 2018

(54) FORMULATIONS FOR MITIGATING OPIOID OVERDOSE AND METHODS OF MAKING AND USING THE SAME

(71) Applicant: Michael Ballek, Raleigh, NC (US)

(72) Inventor: Michael Ballek, Raleigh, NC (US)

(21) Appl. No.: 15/712,756

(22) Filed: Sep. 22, 2017

Related U.S. Application Data
(60) Provisional application No. 62/439,711, filed on Dec. 28, 2016.

(51) Int. Cl. A61K 31/485; A61K 9/00; A61K 9/16

(52) U.S. Cl. CPC A61K 31/485; A61K 9/16; A61K 9/0019

(57) ABSTRACT

The presently disclosed subject matter is directed to opioid antagonist formulations for use in mitigating opioid abuse and/or overdose in a subject. Particularly, the disclosed formulation comprises an immediate release opioid antagonist coupled with an extended release opioid antagonist. The disclosed opioid antagonists attenuate the activity of an opioid agonist and thus prevent or reverse the adverse effects of the agonist. Particularly, the immediate release component allows for immediate availability of the opioid antagonist to provide fast onset treatment, while the extended release component allows continued release of the opioid antagonist to further mitigate the opioid overdose. Thus, the disclosed formulation advantageously provides a fast-onset opioid antagonist in addition to a slow-release opioid antagonist, lasting up to 48 hours post administration.

### CROSS REFERENCE TO RELATED APPLICATIONS

This application is a non-provisional of U.S. Provisional Application No. 62/439,711 filed Dec. 28, 2016 titled “FORMULATIONS FOR MITIGATING OPIOID OVERDOSE AND METHODS OF MAKING AND USING THE SAME”, which is herein incorporated by reference in its entirety.

### TECHNICAL FIELD

The presently disclosed subject matter is directed to formulations for use in mitigating opioid overdose in a subject, and to methods of making and using the disclosed formulations.

### BACKGROUND

The United States is in the midst of an uprecedented opioid epidemic leading to drug overdoses and deaths that have been rising at an alarming rate since 1999. The U.S. Government and the Department of Health and Human Services have developed Opioid Initiative targets to help address the opioid epidemic. The subject matter described herein is an important strategy in helping to combat the drug overdose and deaths that result in exposure to increasingly more potent and longer acting opioids that are readily available and abused in the United States. It is critical to bring forward solutions in a timely fashion to prevent further devastation and death resulting from opioid abuse.

Opioids are a class of drugs that include both natural and synthetic substances. The natural opioids include opium and morphine. Heroin, the most abused opioid, is synthesized from opium. Synthetic opioids include codeine, oxycodone (OXYCONTIN®), meperidine (DEMEROL®), fentanyl (SUBLIMAZE®), hydromorphone (DILAUDID®), methadone and propoxyphene (DARVON®). Opioids, whether used in a clinical or non-clinical environment, are highly addictive and can lead to varying degrees of opioid toxicity. Opioid antagonists are commonly administered to reverse the effects of opioid intoxication or overdose. Particularly, naltrexone is currently available in the United States as either an immediate release tablet or an extended release formulation (VIVITROL®) that is administered in monthly injections. However, neither formulation is sufficient to treat an unconscious patient suffering from acute withdrawal symptoms, as oral tablets cannot be administered to an unconscious patient. Further, month-long injections lack the fast onset and appropriate duration needed to treat opioid overdose. In addition, the effects of potent opioids (such as fentanyl and carfentayl) are difficult to mitigate with the low doses of antagonists currently used.

One opioid antagonist commonly used is Naloxone, available in the United States as an immediate release injectable solution (EVZIO®) or as an intranasal spray (NARCAN®). Due to the rapid elimination and limited dose contained in each intranasal spray, administration instructions require that doses may need to be additionally administered in an absence of response every 2-3 minutes until emergency medical assistance arrives. Such administration requires that a bystander be available to adequately manage an overdose victim, which is not always feasible. Intranasal and intramuscular injections of naloxone result in rapid peak plasma concentrations obtained in 0.5 hours and 0.38 hours, with a rapid half-life (t_{1/2}) of 2.08 hours and 1.24 hours, respectively (Narcan U.S. Prescribing Information). Similarly, following a single EVZIO® subcutaneous or intramuscular injection, median time to peak plasma concentrations (Tₘₐₓ) of naloxone is reached in 15 minutes (range 5 to 80 minutes), with a mean (±SD) peak plasma concentration (Cₘₐₓ) of 1.24 (±0.64) ng/mL. The mean (±SD) plasma half-life of naloxone is 1.28 (±0.48) hours (EVZIO® U.S. Prescribing Information). In contrast, long acting prescription opioids have Tₘₐₓ and half-lives far greater than the short durations of the existing naloxone and naltrexone formulations typically used to treat opioid overdose.

Therefore, it would be beneficial to provide a formulation and intervention method to mitigate opioid overdose in response to long-acting opioids (including both extended release opioid formulations and methadone).

### SUMMARY

In some embodiments, the presently disclosed subject matter is directed to a pharmacological formulation intended for parenteral administration. The disclosed formulation comprises about 1-50 weight percent of an immediate release opioid antagonist, based on the total weight of the formulation and about 50-99 weight percent of an extended release opioid antagonist, based on the total weight of the formulation. In some embodiments, the formulation further comprises one or more pharmaceutically acceptable binders, diluents, lubricants, flow enhancing agents, glidants, pH modifiers, antioxidants, surfactants, lipids, or combinations thereof, present in an amount of from about 50 weight percent or less, based on the total weight of the formulation. In some embodiments, the immediate release opioid antagonist, extended release opioid antagonist, or both are embedded in, encapsulated in, or attached to microspheres with a diameter of about 0.5-20 μm.

In some embodiments, the presently disclosed subject matter is directed to a method of treating an opioid overdose. The method comprises parenterally administering to a subject in need thereof an effective amount of the disclosed formulation to treat the overdose. In some embodiments, the method further comprises administering a second unit dose of an effective amount of a formulation at least 12 hours after the first formulation is administered. In some embodiments, the second formulation is substantially similar or identical to the first formulation.

In some embodiments, the presently disclosed subject matter is directed to a kit comprising the disclosed formulation contained within a sealed package. In some embodiments, the kit further comprises instructions for use and/or a device for administering the formulation (such as a syringe or auto-injector).

In some embodiments, the formulation comprises immediate release opioid antagonist equivalent to about 0.4-10 mg naloxone and extended release opioid antagonist equivalent to about 1.2-240 mg naloxone.

In some embodiments, the extended release opioid antagonist releases antagonist for up to about 4 hours, 8 hours, 12 hours, or 48 hours.

In some embodiments, the subject is a human. In some embodiments, the human has overdosed on an opioid.

In some embodiments, the immediate release opioid antagonist is selected from naloxone, naltrexone, nalmefene, nalorphine, nalbuphine, naloxoneasinen, methylnaltrexone, ketylcyclazocine, norbinaltoprphimne, naltrindol, 6-β-naloxol, 6-β-naltrexol, pharmaceutically acceptable salts thereof, or combinations thereof. Thus, the immediate release opioid antagonist can be naloxone, naltrexone, pharmaceutically acceptable salts thereof, or combinations thereof.

In some embodiments, the extended release opioid antagonist is selected from naloxone, naltrexone, nalmefene, nalorphine, nalbuphine, naloxoneasinen, methylnaltrexone, ketylcyclazocine, norbinaltoprphimne, naltrindol, 6-β-naloxol, 6-β-naltrexol, pharmaceutically acceptable salts thereof, or combinations thereof. Thus, the opioid antagonist can be naloxone or pharmaceutically acceptable salts, or combinations thereof.

In some embodiments, the parenteral administration is selected from intramuscular administration, subcutaneous administration, intravenous administration, or combinations thereof.

In some embodiments, the opioid comprises hydrocodone, morphine, hydromorphone, oxycodone, oxymorphone, fentanyl, carfentanyl, heroin, codeine, levorphanol, meperidine, methadone, or combinations thereof.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “an antagonist” can include a plurality of such antagonists, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

The presently disclosed subject matter is directed to opioid antagonist formulations for use in mitigating opioid abuse and/or overdose in a subject. Particularly, the disclosed formulation comprises an immediate release opioid antagonist coupled with an extended release opioid antagonist. The term “opioid” as used herein refers to any natural or synthetic compound that binds to specific opioid receptors in the central nervous system and/or peripheral nervous system of a subject (such as a human). The term “antagonist” as used herein refers to a molecule that prevents the activation of a receptor. Therefore, the disclosed opioid antagonists attenuate (e.g., block, inhibit, prevent, and/or compete with) the activity of an opioid agonist and thus prevent or reverse the adverse effects of the agonist. The compositions and delivery systems as disclosed herein are effective at reversing the hypoventilation and/or central nervous system depression occurring with opioid overdose.

In some embodiments, the disclosed formulation is a microsphere formulation (such as, but not limited to, a naloxone microsphere formulation). The term “microsphere” as used herein refers to small, synthetic, spherical particles manufactured from various natural and synthetic materials, including glass, polymer, ceramic material, polystyrene, methylstyrene, acrylic polymer, paramagnetic material, carbon graphite, titanium dioxide, latex, cross-linked dextran, cellulose, nylon, cross-linked micelles, Teflon®, and the like. In some embodiments, the microspheres can be hollow, and can have varying density, sphericity, uniformity, particle size, and particle size distribution. The immediate release opioid antagonist, the extended release opioid antagonist, or both can be attached to the microspheres using methods well known in the art. See, for example, U.S. Pat. Nos. 5,652,059; 5,194,300; and 5,830,507, the entire disclosures of which are incorporated by reference herein. In some embodiments, the microspheres have a diameter of about 0.5-20 μm, such as about 1-15 μm, 1-10 μm, or 5-10 μm. Thus, the microspheres can have a diameter of at least about (or no more than about) 1, 2, 3, 4, 5, 6, 7, 8, 9, 10, 11, 12, 13, 14, 15, 16, 17, 18, 19, or 20 μm.

Thus, the immediate release opioid antagonist, extended release opioid antagonist, or both can be embedded in, encapsulated in, or attached to microspheres with a diameter of about 0.5-20 μm. Methods of embedding, encapsulating or attaching agents to microspheres are well known in the art. See, for example, U.S. Patent/Pub. Nos. 2008/0069778; U.S. Pat. Nos. 5,672,965; 8,865,220; 7,132,108; 5,652,059; and 5,830,507, the entire contents of which are incorporated herein by reference.

In some embodiments, the immediate release antagonist microspheres can be prepared using the method set forth in Rajampet, Kadap et al., Intl. J. Pharm. Sci. Vol. 5 Suppl. 3 (July 2013) “Formulation and Evaluation of Octreotide Acetate loaded PLGA Microspheres” and/or Karode, N. et al., World Journal of Pharmacy and Pharmaceutical Sciences, Vol. 4 Issue 4 (26 Mar. 2015) “Sustained Release Injectable Formulations: Its Rationale, Recent Progress, and Advancement”, the entire contents of which are incorporated herein by reference herein.

Alternatively or in addition, other commercially available sustained release drug delivery systems can be used to deliver the immediate release opioid antagonist and/or the extended release opioid antagonist. For example, in some embodiments, oil-based injection systems, injectable drug suspensions, supersaturated drug solutions, and/or in situ forming implants can be used. Such delivery systems are well known to those of ordinary skill in the art.

Advantageously, the disclosed formulation includes a combination of immediate release opioid antagonist with an extended release opioid antagonist that allows for effective treatment during cases of opioid overdose in a subject. Particularly, the immediate release component allows for immediate availability of the opioid antagonist to provide fast onset treatment to a subject in need thereof. Since many overdoses result from extended release opioids, the extended release component of the disclosed formulation allows continued release of the opioid antagonist to further mitigate the potential of opioid overdose in acute overdose situations. Thus, the disclosed formulation advantageously provides a fast-onset opioid antagonist in addition to a slow-release opioid antagonist, lasting up to 48 hours post administration. The disclosed formulation prevents opioid overdose relapse and ensures that enough time is given for an overdose victim to reach emergency treatment.

Opioid antagonists suitable for use with the disclosed formulation can include (but are not limited to) naloxone, naltrexone, nalmefene, nalorphine, nalbuphine, naloxoneasinen, methylnaltrexone, ketylcyclazocine, norbinaltoprphimne, naltrindol, 6-β-naloxol, 6-β-naltrexol, and/or pharmaceutically acceptable salts thereof, including naloxone HCl, naloxone HCl dehydrate, or combinations thereof. Thus, in some embodiments, the opioid antagonist is naloxone, naloxone base, or a pharmaceutically acceptable salt thereof, including naloxone HCl, naloxone HCl dehydrate, or combinations thereof. Unless otherwise specified, the term “naloxone,” as used herein, refers to naloxone, naloxone HCl, naloxone HCl dihydrate, any pharmaceutically acceptable salt of naloxone, or combinations thereof. In other aspects, other opioid antagonists, such as naltrexone, nalmefene, nalorphine, nalbuphine, naloxoneazinen, methylnaltrexone, ketylcyclazocine, norbinaltorphimine, naltrindol, 6-β-naloxol, 6-β-naltrexol, or pharmaceutically acceptable salts thereof, can be used.

Naloxone has the molecular formula C₁₉H₂₁NO₄ and is commonly found under the trade name Narcan®. The structure of naloxone is illustrated below:

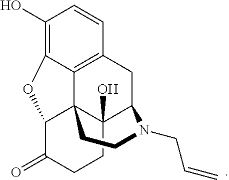

Naloxone is commonly found as the hydrochloride salt (naloxone hydrochloride), a semi-synthetic opiate pure antagonist, differing structurally from oxymorphone only in that the methyl group on the nitrogen atom of oxymorphone is replaced by an allyl group. Further, naloxone hydrochloride is a competitive antagonist at mu, delta, and kappa opioid receptors. Thus, it binds to opioid receptors in the central nervous system in a competitive manner, reversing or inhibiting characteristic opioid effects, including analgesia, sedation, respiratory depression, miosis, bradycardia, and physical dependence. Specifically, naloxone hydrochloride binds to mu opioid receptors with a high affinity, and binds to a lesser degree kappa and gamma opioid receptors.

Opioid antagonists (such as naloxone) prevent or reverse the effects of opioids, including respiratory depression, sedation, and/or hypotension. In addition, the disclosed opioid antagonists have been shown to reverse the psychotomimetic and dysphoric effects of agonist-antagonists, such as pentazocine. When administered in usual doses in the absence of opioids or agonistic effects of other opioid antagonists, naloxone exhibits essentially no pharmacologic activity. Further, naloxone has been shown to avoid tolerance or physical/psychological dependence. In the presence of physical dependence on opioids, naloxone produces withdrawal symptoms. However, in the presence of opioid dependence, withdrawal symptoms appear within minutes of naloxone administration and subside after about 2 hours. The severity and duration of a subject's withdrawal syndrome are related to the dose and route of administration of naloxone, the degree of dependence, and the type of dependence. While not limited to any theory, in vitro evidence suggests that naloxone antagonizes opioid effects by competing for the mu, kappa, and sigma opiate receptor sites in the central nervous system with the greatest affinity for the mu receptor.

The disclosed formulation can have a pH of about 3-7, such as about 3.5-6, or 4-5.5. In some embodiments, the disclosed formulation can have a pH of about 4. Thus, the formulation can have a pH of at least about (or no more than about) 3, 3.25, 3.5, 3.75, 4, 4.25, 4.5, 4.75, 5, 5.25, 5.5, 5.75, 6, 6.25, 6.5, 6.75, or 7.

As set forth above, the disclosed formulation comprises an immediate release (short-acting) opioid antagonist. “Immediate release” as used herein refers to the release of an active ingredient (i.e., opioid antagonist) from a pharmaceutical formulation where the rate of release is not retarded and where the components of the pharmaceutical formulation are designed such that the maximum exposure of the active pharmaceutical ingredient to body tissues occurs in the minimum period of time. In some embodiments, the immediate release component releases opioid antagonist in less than about 1 hour, 45 minutes, or 30 minutes. Thus, the immediate release component can release opioid antagonist in about 2, 3, 4, 5, 10, 15, 20, 25, 30, 35, 40, 45, 50, 55 or 60 minutes or less.

In some embodiments, the immediate release component comprises a unit dose of one or more opioid antagonists equivalent to about 0.4 to 10.0 mg naloxone, such as at least (or no more than) about 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mg. In some embodiments, the immediate release component comprises about 1-50 weight percent of the formulation, such as about 5-45, 10-40, 15-35, or 20-30 weight percent, based on the total weight of the formulation. In some embodiments, the immediate release component comprises about 1, 5, 10, 15, 20, 25, 30, 35, 40, 45, or 50 weight percent of the formulation, based on the total weight of the formulation. It should be appreciated that the weight percent ranges include all numbers in between the recited ranges (i.e., 11, 12, 13, and 14, in between 10 and 15 weight percent).

In some embodiments, the immediate release component of the disclosed formulation can be substantially free of excipient to ensure that the absorption or bioavailability of the opioid antagonist is not slowed after administration.

As set forth above, the disclosed formulation further comprises an extended release (long-acting) opioid antagonist. The term “extended release” as used herein refers to a pharmaceutical formulation that is administered to a patient and has a mechanism to delay the release an active ingredient (i.e., opioid antagonist). In some embodiments, the extended release component of the disclosed formulation includes one or more excipients to slow release and/or bioavailability of the opioid antagonist. Suitable excipients would be well known to those of ordinary skill in the art. Alternatively or in addition, the extended release component can be in the form of a suspension or can include any other mechanism that would function to slow release and/or bioavailability of the opioid antagonist after administration.

In some embodiments, the extended release component comprises a unit dose of one or more opioid antagonists equivalent to about 1.2-240 mg naloxone, such as at least (or no more than) about 1.2, 1.5, 1.8, 2.0, 2.4, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 mg. Thus, in some embodiments, the extended release component comprises a unit dose of one or more opioid antagonists equivalent to about 1.2-240, 2-225, 5-200, 15-175, 20-150, 25-125, 30-100, 35-90, or 40-80 mg naloxone.

In some embodiments, the extended release occurs over a period of at least about 4 hours, such as about 4-48 hours (e.g., 4, 8, 12, 24, 36, 40, or 48 hours). Thus, the extended release can occur over a period of at least (or no more than) about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours. In some embodiments, the final formulations can comprise a weight range of immediate release naloxone microspheres combined with a weight range of extended release naloxone microspheres that meet the desired in vitro release profile and is optimized to deliver the in vivo release profile.

In some embodiments, the extended release component comprises about 50-99 weight percent of the formulation, such as about 50, 55, 60, 65, 70, 75, 80, 85, 90, 95, or 99 weight percent, based on the total weight of the formulation. In some embodiments, the extended release component comprises about 50-99, 55-95, 60-90, 65-85, or 70-80 weight percent of the formulation, based on the total weight of the formulation.

The disclosed compositions are formulated with a suitable carrier to form a pharmaceutically acceptable formulation. In some embodiments, the carrier can comprise water, saline (e.g., 0.9% sodium chloride solution), dextrose (e.g., 5% dextrose solution), or any other aqueous or non-aqueous carriers suitable for parenteral administration. As used herein, the term “parenteral administration” refers to the administration of a composition to a subject via injection or infusion, such as by intravenous injection (into a vein), subcutaneous injection (under the skin), and/or intramuscular injection (into the muscle). In some embodiments, the disclosed formulation can be supplied as single dose vials (e.g., 1 mL each) or as multi-dose vials (e.g., 4 mg/10 mL). When administered intravenously, the disclosed formulation can exhibit an onset of action apparent within about 2 minutes, which is desirable in emergency situations. The onset of action is slightly less rapid when the formulation is administered subcutaneously or intramuscularly. The duration of action is dependent upon the dose and route of administration, as would be appreciated by those of skill in the art.

In addition to the immediate release and extended release components described above, the disclosed formulation can comprise one or more optional components. Suitable optional components can be selected from one or more pharmaceutically acceptable buffers, diluents, lubricants, flow enhancing agents, glidants, pH modifiers, antioxidants, surfactants, lipids, and the like. The disclosed optional components are well known to those of ordinary skill in the art. The optional components can be present in the disclosed formulation in an amount of about 50 wt % or less, such as about 50, 45, 40, 35, 30, 25, 20, 15, 10, 9, 8, 7, 6, 5, 4, 3, 2, 1, 0.5, or 0.1 weight percent, based on the total weight of the formulation.

In some embodiments, the disclosed formulations are stable at room temperature (25° C./60% RH) for at least about 6 months, 9 months, or 12 months. In some embodiments, the disclosed formulations are stable at 40° C./75% RH for at least about 6 months, 9 months, or 12 months. In some embodiments, the disclosed formulations are stable for at least 24-36 months at room temperature (25° C./60% RH).

In some embodiments, the disclosed formulation is sterile and suitable for parenteral administration. The term “sterile” as used herein refers to a pharmaceutical product that is devoid of viable bacteria, fungi, or other microorganisms. The disclosed formulation can be sterilized using any method known or used in the art, including (but not limited to) sterile filtration, irradiation, heat, autoclave, or combinations thereof.

In some embodiments, the disclosed composition can be formulated to be adaptable for use in a manual syringe and/or auto-injector for administration to a subject. One of ordinary skill in the art would be familiar with syringe and auto-injector technology.

Methods of using the disclosed formulations are included within the scope of the presently disclosed subject matter. The methods and compositions disclosed herein are suitable for use by both medical and non-medical personnel.

In some embodiments, the presently disclosed subject matter is directed to methods of treating a known or suspected opioid overdose in a subject in need thereof, wherein the disclosed formulation is administered parenterally. The phrase “subject in need thereof” refers to a subject experiencing or susceptible to an opioid overdose. In some embodiments, the disclosed methods include the complete or partial reversal of opioid intoxication, comprising parenterally administering the disclosed formulation. In some embodiments, the opioid is selected from the group comprising hydrocodone, morphine, hydromorphone, oxycodone, oxymorphone, fentanyl, carfentanyl, heroin, codeine, levorphanol, meperidine, methadone, or combinations thereof.

As set forth herein above, an effective amount of the disclosed formulation can be administered to treat an opioid overdose in a subject in need thereof. The term “effective amount” refers to the amount of the formulation that will be effective in the treatment of a particular subject, and will depend on the particular subject and state of the subject. In some embodiments, the effective amount is the amount of immediate release opioid antagonist and/or extended release opioid antagonist needed to elicit the desired biological response following administration, such as (but not limited to) sufficient blockade of the mu opioid receptor. It should be appreciated that the effective amount can be determined by standard clinical techniques. In addition, in vitro or in vivo assays can optionally be employed to help identify optimal dosage ranges for a particular subject. In some embodiments, the subject can be administered an effective dose of a formulation comprising immediate release opioid antagonist equivalent to about 0.4-10 mg naloxone and extended release opioid antagonist equivalent to about 1.2-240 mg naloxone.

In some embodiments, the disclosed method can comprise a second administration of the disclosed formulation to a subject in need thereof at least 4 hours (i.e., 4, 5, 6, 7, 8, 9, 10, 11, 12, 13, 14, 15, 16, 17, 18, 19, 20, 21, 22, 23, or 24 hours) after administration of the first formulation. In some embodiments, the formulation used in the second administration is substantially similar to the formulation used in the first administration. As used herein, “substantially similar” refers to dissolution profiles that have a similar factor score of 50-100, as would be known to those of ordinary skill in the art. In some embodiments, the formulation used in the second administration is identical to the formulation used in the first administration.

In some embodiments, the known or suspected opioid overdose can be manifested by respiratory and/or central nervous system depression. Thus, in some embodiments, the disclosed formulation can be administered parenterally to partially or completely reverse opioid intoxication. It should be appreciated that the phrase “treating an opioid overdose” includes reversing the effects of an opioid overdose.

In some embodiments, the presently disclosed subject matter can encompass kits that can simplify the administration of the disclosed formulation. A typical kit can comprise a unit dosage form of an immediate release opioid antagonist and a unit dosage form of an extended release opioid antagonist, or a combination thereof. The disclosed kit can further include a label or printed instructions regarding use of the formulation to treat the opioid overdose. In some embodiments, the kit comprises a device that is useful for administering the unit dosage formulation, such as (but not limited to) a syringe or auto-injector.

The presently disclosed subject matter can be practiced with human subjects, and is applicable to both males and females, without age restrictions. The presently disclosed subject matter can also be practiced in non-human subjects, such as laboratory animals.

### EXAMPLES

The following Examples provide illustrative embodiments. In light of the present disclosure and the general level of skill in the art, those of ordinary skill in the art will appreciate that the following Examples are intended to be exemplary only and that numerous changes, modifications, and alterations can be employed without departing from the scope of the presently disclosed subject matter.

### Prophetic Example 1

### Manufacturing Method for Immediate and Extended Release Naloxone HCl

It would be beneficial to provide an immediate release component equivalent to 0.4-10 mg naloxone and an extended release naloxone equivalent to about 1.2-240 mg naloxone. It would further be beneficial to provide a formulation with an extended release profile that provides naloxone up to or about 4 hours, 8 hours, 12 hours or 48 hours. To achieve these goals, a formulation will be prepared that includes a weight range of immediate release naloxone microspheres combined with a weight range of extended release naloxone microspheres that meet the desired in vitro release profile and is optimized to deliver the in vivo release profile.

The design of experiments will be closely patterned to the publication Rajampet, Kadap et al., Intl. J. Pharm. Sci. Vol. 5 Suppl. 3 (July 2013) “Formulation and Evaluation of Octreotide Acetate loaded PLGA Microspheres”, the entire contents of which are incorporated herein by reference herein. The molecular weights of the polymers reflect the disclosure set forth in Karode, N. et al. World Journal of Pharmacy and Pharmaceutical Sciences, Vol. 4, Issue 4, (26 Mar. 2015) “Sustained Release Injectable Formulations: Its Rationale, Recent Progress, and Advancement”, the entire content of which is incorporated herein by reference.

The manufacturing method will follow the general method set forth in Karode, N. et al. In comparison to the water/oil (w/o) method, it is believed that the water/oil/water (w/o/w) method is more suitable for water soluble drugs. The cited method will be used to load microparticles with extended release opioid antagonist and/or immediate release opioid antagonist (such as naloxone, naltrexone, nalmefene, nalorphine, nalbuphine, naloxoneasinen, methylnaltrexone, ketylcyclazocine, norbinaltoprphimne, naltrindol, 6-β-naloxol, 6-β-naltrexol, pharmaceutically acceptable salts thereof, or combinations thereof) with high yield and encapsulation efficiency. As set forth in Karode, the method includes the steps of: mixing an aqueous drug solution or dispersion with PGLA solution in organic solvent. A large amount of water containing emulsifier is then mixed into the drug solution/PGLA/organic solvent mixture. The solvent is then removed using a w/o/w method. The microsphere product is then washed and filled into suitable containers. The microsphere product can then be dried using standard freeze drying or lyophilize methods known in the art.

The method includes the following variables that will be determined: pH 3.0-6.5 (pH; volume), solvent phase (methylene chloride) volume, counter solvent (ethyl acetate) volume, emulsifier (1% polyvinyl alcohol aqueous solution) volume, high homogenization speed (10k rpm), short homogenization time (1 min), low homogenization speed (6k rpm), long homogenation time (3 min), and stirring speed (1k rpm). The formulation variables will be tested according to Table 1 (short duration formulation 1), Table 2 (short duration formulation 1.1), Table 3 (short duration formulation 2), Table 4 (short duration formulation 2.1), Table 5 (long duration formulation A), Table 6 (long duration formulation A.1), Table 7 (long duration formulation B), Table 8 (long duration formulation B.1), Table 9 (short duration formulation 3), Table 10 (short duration formulation 3.1), Table 11 (short duration formulation 4); Table 12 (short duration formulation 4.1), Table 13 (long duration formulation C), Table 14 (long duration formulation C.1), Table 15 (long duration formulation D), and Table 16 (long duration formulation D.1).

Thus, as set forth in Tables 1-8 (below), a total of 64 different formulations will be prepared. An additional 64 formulations would change the following: hold 1% PVA aqueous solution constant at 100 mL, change high homogenation speed to 6k rpm (6,000 rpm) for replicate 1 and 14k rpm for replicate 3, change short homogenation time to 3 min for replicate 4 and 4 min for replicate 5, and change stirring temperature to 25° C. for replicate 7 and 40° C. for replicate 8.

As set forth in Tables 9-16, 64 different formulations (short and long) with higher naloxone load were prepared. An additional 64 formulations would change the following: change naloxone HCl load from 10.7% w/w to 19.35% w/w to 32.4% w/w to 50% w/w, hold 1% PVA aqueous solution constant at 100 mL, change high homogenation speed to 6k for #1 and 14k for #3, change short homogenation time to 3 min for #4 and 5 min for #5, and change stirring temperature to 25° C. for #7 and 40° C. for #8. Naloxone doses represented are examples and may be changed within the ranges specified.

TABLE 1*
Short Duration Formulation 1

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 0.4 | 0.4 | 0.4 | 0.4 | 0.4 | 0.4 | 0.4 | 0.4 |
| Buffer pH 3.0-6.5 | 1 | 1 | 1 | 1 | 1 | 0.5 | 1.5 | 1 |
| PLGA 50:50 (mw 45 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| High homogenization | 10k | 10k | 10k | 10k | 10k | 10k | 10k | 10k |
| speed (rpm) | | | | | | | | |
| Short homogenization | 1 | 1 | 1 | 1 | 1 | 1 | 1 | 1 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

*Doses or Naloxone HCl may be further adjusted as per specified ranges.

TABLE 2
Short Duration Formulation 1.1

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 0.4 | 0.4 | 0.4 | 0.4 | 0.4 | 0.4 | 0.4 | 0.4 |
| Buffer pH 3.0-6.5 | 1 | 1 | 1 | 1 | 1 | 0.5 | 1.5 | 1 |
| PLGA 50:50 (mw 45 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| Low homogenation | 6k | 6k | 6k | 6k | 6k | 6k | 6k | 6k |
| speed (rpm) | | | | | | | | |
| Short homogenation | 3 | 3 | 3 | 3 | 3 | 3 | 3 | 3 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 3
Short Duration Formulation 2

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 0.4 | 0.4 | 0.4 | 0.4 | 0.4 | 0.4 | 0.4 | 0.4 |
| Buffer pH 3.0-6.5 | 1 | 1 | 1 | 1 | 1 | 0.5 | 1.5 | 1 |
| PLGA 50:50 (mw 74 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| High homogenation | 10k | 10k | 10k | 10k | 10k | 10k | 10k | 10k |
| speed (rpm) | | | | | | | | |
| Short homogenation | 1 | 1 | 1 | 1 | 1 | 1 | 1 | 1 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 4
Short Duration Formulation 2.1

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 0.4 | 0.4 | 0.4 | 0.4 | 0.4 | 0.4 | 0.4 | 0.4 |
| Buffer pH 3.0-6.5 | 1 | 1 | 1 | 1 | 1 | 0.5 | 1.5 | 1 |
| PLGA 50:50 (mw 74 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| Low homogenation | 6k | 6k | 6k | 6k | 6k | 6k | 6k | 6k |
| speed (rpm) | | | | | | | | |
| Short homogenation | 3 | 3 | 3 | 3 | 3 | 3 | 3 | 3 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 5
Long Duration Formulation A

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 1.2 | 1.2 | 1.2 | 1.2 | 1.2 | 1.2 | 1.2 | 1.2 |
| Buffer pH 3.0-6.5 | 1 | 1 | 1 | 1 | 1 | 0.5 | 1.5 | 1 |
| PLGA 75:25 (mw 54 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| High homogenation | 10k | 10k | 10k | 10k | 10k | 10k | 10k | 10k |
| speed (rpm) | | | | | | | | |
| Long homogenation | 1 | 1 | 1 | 1 | 1 | 1 | 1 | 1 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 6
Long Duration Formulation A.1

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 1.2 | 1.2 | 1.2 | 1.2 | 1.2 | 1.2 | 1.2 | 1.2 |
| Buffer pH 3.0-6.5 | 1 | 1 | 1 | 1 | 1 | 0.5 | 1.5 | 1 |
| PLGA 75:25 (mw 54 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| Low homogenation | 6k | 6k | 6k | 6k | 6k | 6k | 6k | 6k |
| speed (rpm) | | | | | | | | |
| Long homogenation | 3 | 3 | 3 | 3 | 3 | 3 | 3 | 3 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 7
Long Duration Formulation B

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 1.2 | 1.2 | 1.2 | 1.2 | 1.2 | 1.2 | 1.2 | 1.2 |
| Buffer pH 3.0-6.5 | 1 | 1 | 1 | 1 | 1 | 0.5 | 1.5 | 1 |
| PLGA 75:25 (mw 65 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| High homogenation | 10k | 10k | 10k | 10k | 10k | 10k | 10k | 10k |
| speed (rpm) | | | | | | | | |
| Long homogenation | 1 | 1 | 1 | 1 | 1 | 1 | 1 | 1 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 8
Long Duration Formulation B.1

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 1.2 | 1.2 | 1.2 | 1.2 | 1.2 | 1.2 | 1.2 | 1.2 |
| Buffer pH 3.0-6.5 | 1 | 1 | 1 | 1 | 1 | 0.5 | 1.5 | 1 |
| PLGA 75:25 (mw 54 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| Low homogenation | 6k | 6k | 6k | 6k | 6k | 6k | 6k | 6k |
| speed (rpm) | | | | | | | | |
| Long homogenation | 3 | 3 | 3 | 3 | 3 | 3 | 3 | 3 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 9
Short Duration Formulation 3

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 10 | 10 | 10 | 10 | 10 | 10 | 10 | 10 |
| Buffer pH 3.0-6.5 | 1 | 1 | 1 | 1 | 1 | 0.5 | 1.5 | 1 |
| PLGA 50:50 (mw 45 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| High homogenation | 10k | 10k | 10k | 10k | 10k | 10k | 10k | 10k |
| speed (rpm) | | | | | | | | |
| Short homogenation | 1 | 1 | 1 | 1 | 1 | 1 | 1 | 1 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 10
Short Duration Formulation 3.1

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 10 | 10 | 10 | 10 | 10 | 10 | 10 | 10 |
| Buffer pH 3.0-6.5 | 1 | 1 | 1 | 1 | 1 | 0.5 | 1.5 | 1 |
| PLGA 50:50 (mw 45 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| low homogenation | 6k | 6k | 6k | 6k | 6k | 6k | 6k | 6k |
| speed (rpm) | | | | | | | | |
| long homogenation | 3 | 3 | 3 | 3 | 3 | 3 | 3 | 3 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 11
Short Duration Formulation 4

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 10 | 10 | 10 | 10 | 10 | 10 | 10 | 10 |
| Buffer pH 3.0-6.5 | 1 | 1 | 1 | 1 | 1 | 0.5 | 1.5 | 1 |
| PLGA 50:50 (mw 74 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| High homogenation | 10k | 10k | 10k | 10k | 10k | 10k | 10k | 10k |
| speed (rpm) | | | | | | | | |
| Short homogenation | 1 | 1 | 1 | 1 | 1 | 1 | 1 | 1 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 12
Short Duration Formulation 4.1

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 10 | 10 | 10 | 10 | 10 | 10 | 10 | 10 |
| Buffer pH 3.0-6.5 | 1 | 1 | 1 | 1 | 1 | 0.5 | 1.5 | 1 |
| PLGA 50:50 (mw 74 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| Low homogenation | 6k | 6k | 6k | 6k | 6k | 6k | 6k | 6k |
| speed (rpm) | | | | | | | | |
| Long homogenation | 3 | 3 | 3 | 3 | 3 | 3 | 3 | 3 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 13
Long Duration Formulation C

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 240 | 240 | 240 | 240 | 240 | 240 | 240 | 240 |
| Buffer pH 3.0-6.5 | 1 | 1 | 1 | 1 | 1 | 0.5 | 1.5 | 1 |
| PLGA 75:25 (mw 54 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| High homogenation | 10k | 10k | 10k | 10k | 10k | 10k | 10k | 10k |
| speed (rpm) | | | | | | | | |
| Short homogenation | 1 | 1 | 1 | 1 | 1 | 1 | 1 | 1 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 14
Long Duration Formulation C.1

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 240 | 240 | 240 | 240 | 240 | 240 | 240 | 240 |
| Buffer pH 3.0-6.5 | 1 | 1 | 1 | 1 | 1 | 0.5 | 1.5 | 1 |
| PLGA 75:25 (mw 54 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| Low homogenation | 6k | 6k | 6k | 6k | 6k | 6k | 6k | 6k |
| speed (rpm) | | | | | | | | |
| Long homogenation | 3 | 3 | 3 | 3 | 3 | 3 | 3 | 3 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 15
Long Duration Formulation D

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 240 | 240 | 240 | 240 | 240 | 240 | 240 | 240 |
| Buffer pH 3.0-6.5 | 1 | 1 | 1 | 1 | 1 | 0.5 | 1.5 | 1 |
| PLGA 75:25 (mw 65 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| High homogenation | 10k | 10k | 10k | 10k | 10k | 10k | 10k | 10k |
| speed (rpm) | | | | | | | | |
| Short homogenation | 1 | 1 | 1 | 1 | 1 | 1 | 1 | 1 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

TABLE 16
Long Duration Formulation D.1

| | Formulation Variables | | | | | | | |
|---|---|---|---|---|---|---|---|---|
| Composition | #1 | #2 | #3 | #4 | #5 | #6 | #7 | #8 |
| Naloxone HCl (mg) | 240 | 240 | 240 | 240 | 240 | 240 | 240 | 240 |
| Buffer pH 3.0-6.5 | 1 | 1 | 1 | 1 | 1 | 0.5 | 1.5 | 1 |
| PLGA 75:25 (mw 54 kDa) | 500 | 500 | 500 | 500 | 500 | 500 | 500 | 500 |
| Methylene chloride (mL) | 2.5 | 5 | 7.5 | 5 | 5 | 5 | 5 | 5 |
| Ethyl acetate (mL) | 0 | 0 | 0 | 0 | 0 | 0 | 0 | 5 |
| Polyvinyl alcohol, 1% | 100 | 100 | 100 | 50 | 150 | 100 | 100 | 100 |
| aqueous solution (mL) | | | | | | | | |
| Low homogenation | 6k | 6k | 6k | 6k | 6k | 6k | 6k | 6k |
| speed (rpm) | | | | | | | | |
| Long homogenation | 3 | 3 | 3 | 3 | 3 | 3 | 3 | 3 |
| time (min) | | | | | | | | |
| Homogenization | 5 | 5 | 5 | 5 | 5 | 5 | 5 | 5 |
| temperature (° C.) | | | | | | | | |
| Stirring speed (rpm) | 1k | 1k | 1k | 1k | 1k | 1k | 1k | 1k |
| Stirring time (hours) | 2 | 2 | 2 | 2 | 2 | 2 | 2 | 2 |
| Stirring temperature (° C.) | 5 | 5 | 5 | 5 | 5 | 5 | 5 | 5 |

### Prophetic Example 2

### In Vitro Evaluation of Formulation Replicates

The different formulations produced in Example 1 will be evaluated to determine yield (practical yield/theoretical yield×100%), drug loading (actual weight of drug in sample/theoretical weight in sample×100%), particle size analysis (laser diffractometer) suspension in aqueous surfactant, scanning electron microscope (SEM), in vitro drug release (USP dissolution apparatus with PBS pH 7.4), stability (3 months accelerated and controlled room temperature per ICH guidelines), syringeability (in same vehicle as Vivitrol®, the composition of the diluent includes carboxymethylcellulose sodium salt, polysorbate 20, sodium chloride, and water for injection).

Successful candidates will then undergo sterilization experiments to determine robustness of microspheres (steam autoclave, gamma irradiation, electron beam irradiation). Microspheres will be evaluated for particle size and in vitro drug release.

What is claimed is:

1. A pharmacological formulation intended for parenteral administration, said formulation comprising:
a. about 1-50 weight percent of an immediate release opioid antagonist, based on the total weight of the formulation; and
b. about 50-99 weight percent of an extended release opioid antagonist, based on the total weight of the formulation.

2. The formulation of claim 1, wherein the formulation comprises immediate release opioid antagonist equivalent to about 0.4-10 mg naloxone and extended release opioid antagonist equivalent to about 1.2-240 mg naloxone.

3. The formulation of claim 1, wherein the immediate release opioid antagonist, extended release opioid antagonist, or both are embedded in, encapsulated in, or attached to microspheres with a diameter of about 0.5-20 μm.

4. The formulation of claim 1, wherein the extended release opioid antagonist releases antagonist for up to about 4, 12, or 48 hours.

5. The formulation of claim 1, further comprising one or more pharmaceutically acceptable binders, diluents, lubricants, flow enhancing agents, glidants, pH modifiers, antioxidants, surfactants, lipids, or combination thereof.

6. The formulation of claim 1, wherein the immediate release opioid antagonist, extended release opioid antagonist, or both are selected from naloxone, naltrexone, pharmaceutically acceptable salts thereof, or combinations thereof.

7. The formulation of claim 1, wherein the parenteral administration is selected from intramuscular administration, subcutaneous administration, intravenous administration, or combinations thereof.

8. A kit comprising a formulation that is contained within a sealed package, wherein the formulation comprises:
a. about 1-50 weight percent of an immediate release opioid antagonist, based on the total weight of the formulation; and
b. about 50-99 weight percent of an extended release opioid antagonist, based on the total weight of the formulation.

9. The kit of claim 8, further comprising instructions for use, a device for administering the formulation, or both.

10. The kit of claim 8, wherein the formulation comprises immediate release opioid antagonist equivalent to about 0.4-10 mg naloxone and extended release opioid antagonist equivalent to about 1.2-240 mg naloxone.

11. The kit of claim 8, wherein the immediate release opioid antagonist, extended release opioid antagonist, or both are embedded in, encapsulated in, or attached to microspheres with a diameter of about 0.5-20 μm.

12. The kit of claim 8, wherein extended release opioid antagonist releases antagonist for up to about 12 or 48 hours.

13. The kit of claim 8, wherein the immediate release opioid antagonist, extended release opioid antagonist, or both are selected from naloxone, naltrexone, pharmaceutically acceptable salts thereof, or combinations thereof.

14. A method of treating an opioid overdose in a subject, the method comprising parenterally administering to a subject in need thereof an effective amount of a formulation to treat the overdose, wherein the formulation comprises:
a. about 1-50 weight percent of an immediate release opioid antagonist, based on the total weight of the formulation; and
b. about 50-99 weight percent of an extended release opioid antagonist, based on the total weight of the formulation.

15. The method of claim 14, wherein the opioid is selected from hydrocodone, morphine, hydromorphone, oxycodone, oxymorphone, fentanyl, carfentanyl, heroin, codeine, levorphanol, meperidine, methadone, or combinations thereof.

16. The method of claim 14, wherein the formulation comprises immediate release opioid antagonist equivalent to about 0.4-10 mg naloxone and extended release opioid antagonist equivalent to about 1.2-240 mg naloxone.

17. The method of claim 14, wherein the extended release opioid antagonist releases antagonist for up to about 4, 12, or 48 hours.

18. The method of claim 14, wherein the immediate release opioid antagonist, extended release opioid antagonist, or both are selected from naloxone, naltrexone, pharmaceutically acceptable salts thereof, or combinations thereof.

19. The method of claim 14, wherein the parenteral administration is selected from intramuscular administration, subcutaneous administration, intravenous administration, or combinations thereof.

20. The method of claim 14, further comprising administering a second unit dose of an effective amount of a formulation at least 12 hours after the first formulation is administered.
